(19) United States
(12) Patent Application Publication
(10) Pub. No.: US 2020/0060981 A1
(43) Pub. Date: Feb. 27, 2020

(54) CATIONIC NANOPARTICLES FOR ENHANCING INFECTIOUS CAPACITY OF LIVE VIRUSES

(71) Applicants: UNIVERSITE DE LILLE, Lille (FR); CENTRE HOSPITALIER REGIONAL ET UNIVERSITAIRE DE LILLE (CHRU), Lille (FR); UNIVERSITE D'ARTOIS, Arras (FR)

(72) Inventors: Didier Betbeder, Houplin-Ancoisne (FR); Didier Hober, Camphin-en-Carembault (FR)

(21) Appl. No.: 16/467,188

(22) PCT Filed: Dec. 11, 2017
(86) PCT No.: PCT/EP2017/082151

§ 371 Date: Jun. 6, 2019

(30) Foreign Application Priority Data
Dec. 9, 2016 (IB) PCT/IB2016/002010

(51) Int. Cl. A61K 9/51; C12N 7/00; A61K 39/13; A61K 39/155; A61K 39/23; A61K 39/245; A61K 48/00; A61K 9/127; A61P 31/14; A61P 31/20; A61P 31/22

(52) U.S. Cl. CPC A61K 9/5161; A61K 2039/5254; A61K 39/13; A61K 39/155; A61K 39/23; A61K 39/245; A61K 48/0041; A61K 9/5153; A61K 9/1272; A61P 31/14; A61P 31/20; A61P 31/22; C12N 2770/32645; C12N 2750/14345; C12N 2720/12345; C12N 2710/16645; C12N 2770/24345; C12N 2770/16045; C12N 2760/18545; C12N 7/00

(57) ABSTRACT

A combination of cationic nanoparticles and viruses and uses thereof. The use of nanoparticles for enhancing the infectious capacity of a live virus, preferably a non-enveloped live virus.

### FIELD OF THE INVENTION

The present invention relates to a combination of cationic nanoparticles and viruses and uses thereof. The invention particularly relates to the use of nanoparticles for enhancing the infectious capacity of a live virus, preferably a non-enveloped live virus.

### BACKGROUND OF THE INVENTION

Although being responsible for millions of death every year throughout the world, viruses are also considered as promising tools in the prevention and the treatment of several diseases. Viruses efficiently gain access to host cells and exploit their cellular machinery to facilitate their replication. These capacities make them appear as very interesting tools for targeting and acting on a specific group of cells such as diseased cells or can be used for vaccination. The concept of virotherapy harnesses the viral infection but avoid the subsequent expression of viral genes that leads to replication and toxicity. Virotherapy is a treatment using biotechnology to convert viruses into therapeutic agents by reprogramming viruses to treat diseases. There are three main branches of virotherapy: anti-cancer oncolytic viruses, viral vectors for gene therapy and viral immunotherapy. These three approaches can be gathered under the term of «modified virus-based therapy».

Today, modified viruses are used for treating pathologies such as cancer, cardiovascular diseases, neurodegenerative disorders and infectious disease (see Thomas et al, Nature Reviews Genetics, 2003, vol. 4, 346-358). However, such viruses have a restricted infectious capacity and large quantities of viruses are usually required for obtaining a therapeutic effect.

Thus, there is a need to develop novel pathways for improving the infectious capacity of modified viruses, particularly for potentiating their therapeutic efficiency or for improving their production yield.

The publication of Nittaya et al. “Effects of nanoparticles coating on the activity of oncolytic adenovirus-magnetic nanoparticle complexes” describes effect of magnetic nanoparticles on adenovirus infectivity. The publication detailed three core-shell-type iron oxide magnetic nanoparticles differing on their surface coatings, particles sizes and magnetic properties for their ability to enhance the oncolytic potency of specific adenovirus and to stabilize it against the inhibitory effects of serum or a neutralizing antibody. The use of magnetic nanoparticles in a magnetic field limits the applications of such technology.

The inventors of the present invention have now discovered that it is possible to increase the capacity of live virus, especially of non-enveloped live viruses to infect cells by simply combining them with specific nanoparticles even in presence of serum.

### SUMMARY OF THE INVENTION

The present invention relates to the use of nanoparticles for enhancing the infectious capacity of a live virus. Thus, the present invention relates to cationic nanoparticles combined with live viruses, uses thereof, a method for preparing a combination of nanoparticles with live viruses and a method for producing live viruses. Furthermore, the invention also concerns the use of such combination in medical applications, including treatment of cancer, cardiovascular diseases, neurodegenerative disorders and infectious diseases, as well as vaccines, gene therapy and virus preparation stabilisation.

### DETAILED DESCRIPTION OF THE INVENTION

The present inventors have discovered that it is possible to enhance the infectious capacity of live virus, especially non-enveloped viruses by combining them with nanoparticles, specifically with cationic nanoparticles. Particularly, they have observed that non-enveloped live viruses combined with cationic nanoparticles were able to infect cells at a much lower concentration than that necessary for non-combined viruses, also called free viruses. This ability is particularly interesting in viral production, for vaccines or in viral therapies, including gene therapy approaches, where large quantities of viruses are currently required for obtaining a desired effect.

Thus, in a first aspect, the present invention relates to the use of a cationic nanoparticle for enhancing the infectious capacity of a live virus.

In the context of the present invention, “the infectious capacity of a virus” refers to the capacity of a virus to infect cells. In a preferred embodiment, the infectious capacity of the virus is enhanced by 10-fold, more preferably 100-fold, and even more preferably 1000-fold. The improvement of the infectious capacity is linked to particular combinations of a virus type with a particular cationic nanoparticle type. Examples of such improvements are provided under 3 b) of the results section and FIG. 7.

As used herein, “live virus” refers to a virus that is not killed. A live virus can enter and replicate into permissive cells or should retains one of these properties: the ability to enter into the cells, to replicate and/or to be inserted in the genome. A live virus can be attenuated or not, defective or not, and recombinant or not. A recombinant virus can for example be modified by the introduction of an exogene to increase or modulate its immunogenicity or to improve protein production. On the contrary, a killed virus has lost all viral functionalities, especially if viral proteins or nucleic acids have been denaturated by any methods, for example by thermal inactivation, by chemical or physical crosslinking, by the use of destabilizing agents such as detergents or any other present and future processes.

Viruses can use different pathways for entering into the cells. One of these pathways consists in entering via endocytosis: the virus binds to a surface receptor present on the cell and is thereby endocytosed into the cell via an endocytic vesicle. The present inventors believe that nanoparticles enhance the infectious capacity of viruses by improving their endocytosis and/or by improving their capacity to escape from endocytic vesicles.

According to the invention, viruses used in combination with nanoparticles are live viruses, such as adenovirus, retrovirus, papillomavirus, parvovirus, bacteriophages, baculovirus and all viruses used in vaccines, gene therapy, oncotherapy or used for recombinant or natural protein production in cells, preferably chosen among non-enveloped viruses. Viruses useful in the invention include DNA viruses and RNA viruses. They may be selected, but are not limited to, among the following families: Adenoviridae, Caulimoviridae, Rudiviridae, Papillomarividae, Phycodnaviridae, Tectiviridae, Papovaviridae, Circoviridae, Parvoviridae, Birnaviridae, Reoviridae, Astroviridae, Caliciviridae, Picornaviridae, Potyviridae, Poliomarividae, Hepeviridae, Arteriviridae, Anelloviridia, Papillomarividae, Paramyxoviridae, Togaviridiae, Herpesviridae, Orthomyxoviridae, Flaviviridae, Hepadnaviridae, Rhabdoviridae, Poxviridae, Filoviridae, Retroviridae, Coronaviridae, Baculoviridae, Reoviridae and phages and bacteriophages such as, but not limited to, Myoviridae, Siphoviridae, Podoviridae.

More specifically, non-enveloped viruses useful in the present invention include, without being limited thereto, Parovirinae, Picornaviridae, Densovirus, Dependovirus, Papillomavirus, Polyomavirus, Mastadenovirus, Adenoviridae, Enterovirus, Hepatovirus, Rhinovirus, Norovirus, Astrovirus, Arterivirus, Orthoreovirus, Orbivirus, Rotavirus, Coltivirus, Avibirnavirus, Birnaviridae and Reovirus. In a preferred embodiment, the non-enveloped virus is selected from the group consisting Enterovirus (Echo, Poliovirus 1), IBDV Gumboro, Rotavirus (rotavirus SA11).

According to the invention, the different types of viruses can be used combined for the different applications described herein.

Examples of viruses used for vaccines are listed below:

| | | |
|---|---|---|
| Paramyxoviridae (Envelope) | → Morbillivirus | → Measles virus |
| | → Rubulavirus | → Mumps virus |
| | → Pneumovirus | → RSV |
| Togaviridiae (Envelope) | →Rubivirus | → Rubella virus |
| | → Alpha virus | → Eastern Equine encephalitis virus |
| | | → Enterovirus 71 |
| Herpesviridae (Envelope) | → Varicellovirus | → Zoster virus |
| | → Herpesvirus | → Herpes simplex |
| | → Cytomegalovirus | |
| | → Lymphocryptovirus | → Epstein-barr virus |
| Orthomyxoviridae (Envelope) | → Influenzavirus | → Influenza virus |
| Reoviridae | → Rotavirus | |
| Flaviviridae (Envelope) | → Flavivirus | → Yellow fever |
| | → Japanese encephalitis virus | |
| | → Tick-borne encephalitis virus | |
| | → Dengue virus | |
| | → West Nile virus | |
| | → Zika virus | |
| | → Hepatitis C virus | |
| Papilliomaviridae | → Papillomavirus | → Human Papillomavirus |
| Hepadnaviridae (envelope) | → Orthohepadnavirus | → Hepatitis B virus |
| Picornaviridae | → Hepatovirus | → Hepatitis A virus |
| | → Enterovirus | → Poliovirus |
| | | → Coxsakie B virus |
| Rhabdoviridae (Envelope) | → Lyssavirus | → Rabies virus |
| Poxviridae (Envelope) | → Orthopoxvirus | → Smallpox virus |
| | → Vaccinia virus | |
| | → Avipoxvirus | → Avipox virus |
| Hepeviridae | → Orthohepevirus | → Hepatitis E |
| Filoviridae (Envelope) | → Ebolavirus | → Ebola virus |
| | → Marburgvirus | → Marburg virus |
| Retroviridae (Envelope) | → Lentivirus | → HIV |
| | → Deltaretrovirus | → HTLV-1 |
| Calciviridae | → Norovirus | |
| Coronaviridae (enveloppe) | → Coronavirus | → SARS |
| Adenoviridae | → Adenovirus | |
| Baculoviridae | → Baculovirus | |
| Bacteriophage (Non-enveloped) | | |
| Siphoviridae | | |
| Myoviridae | | |
| Podoviridae | | |

The virus used is a live virus which is attenuated or not, defective or not, and recombinant or not, preferably a live non-enveloped virus, for example for vaccines, gene therapy, oncotherapy or used for recombinant or natural protein production in cells.

In the meaning of the present invention, nanoparticles are particles having a size range between 1 and 500 nanometers. More preferably, the nanoparticles have a size range between 10 and 300 nm, especially between 30 and 250 nm. They can be made of organic or inorganic material or a mixture of organic and inorganic compound. They can also be porous or not and their surface can be anionic, cationic, neutral (hydrophobic or hydrophilic or a mixture of all these properties). Moreover, a nanoparticle according to the invention is advantageously used in solution. Thus, the term nanoparticle also includes particles or molecules which are in a nanoparticulate form in solution, such as e.g. chitosan. The solution may be an aqueous solution, a buffer solution or a serum solution. The inventors have indeed found that certain linear molecules such as chitosan form nanoscale coils in solution, which behave as conventional nanoparticles. Chitosan may thus be used in the form of a conventional nanoparticle (e.g. Qi et al., Carbohydrate Research, 2004, 339(16), 2693-2700) or as such or as hydrolysate in solution.

According to the invention, the nanoparticles are cationic nanoparticles. Suitable cationic nanoparticles are for example:
Cationic polysaccharide nanoparticles such as cationic maltodextrin nanoparticles or chitosan nanoparticles. Cationic maltodextrin nanoparticles are for example porous maltodextrin nanoparticles with or without a lipid core (see Paillard et al., Pharm Res., 2010, 27(1), 126-133). Maltodextrin nanoparticles without a lipid core correspond to cationic reticulated nanoparticles, also called NP+ in the experimental part. For maltodextrin nanoparticles with a lipid core, the core can for example correspond to dipalmitoyl phosphatidyl glycerol (DG); the resulting nanoparticle is called DGNP (or NPL) in the experimental part. Chitosan nanoparticles can correspond for example to chitosan nanoparticles and their derivatives for example (trimethyl-chitosan) (see Qi et al., Carbohydrate Research, 2004, 339(16), 2693-2700),
Chitosan and their hydrolysates (in solution),
Cationic Poly Lactic Acid (PLA), Poly glycolic acid (PGA) or poly(lactic-co-glycolic acid) (PLGA) nanoparticles which can be for example coated with chitosan (see for instance Kumar et al., Biomaterials, 2004, 25(10), 1771-1777, or Cuiet al, Journal of Controlled Release, 2001, 75(3), 409-419) or with polyethylenimine (PEI), with CTAB (Cetyl TrimethylAmmonium bromide), or with primary, secondary, tertiary or quaternary amine compounds, such as trimethylamoniumchitosan.
Cationic micelles or cationic liposomes (see Gao et al, Biochemical and biophysical research communications, 1991, 179(1), 280-285).

Where the cationic charge of the nanoparticle is obtained via a cationic ligand, this ligand can be covalently linked or adsorbed to the surface of the nanoparticles. For example, the cationic polysaccharide may be a crosslinked polymer and may be obtained by the reaction between a polysaccharide chosen among starch, dextran, dextrin, and maltodextrin preferably, derivatized with cationic ligands such as quaternary ammonium. Primary, secondary and tertiary amines may also be used. Particularly, the cationic polysaccharide can be obtained from the reaction between maltodextrin and glycidyl-trimethyl-ammonium chloride.

In a particularly preferred embodiment, nanoparticles used according to the invention are porous maltodextrin without or with lipid core nanoparticle (NP+ or DGNP, respectively). Such nanoparticles are disclosed, for example, in Paillard et al., (Paillard et al., Pharm Res., 2010, 27(1), 126-133) and in WO2014/041427 and are referred to as ₇₀DGNP⁺ nanoparticles or as DG70 nanoparticles. Porous nanoparticles can also be obtained from chitosan and their derivatives such as trimethyl chitosan. As set forth above, chitosan alone or a hydrolysate thereof may also be used as it forms by itself nanoparticles in solution. Non porous cationic nanoparticles can also be used such as PLA (Poly Lactic Acid) or PGA (Poly glycolic acid) or PLGA (Poly Lactic co-Glycolic acid) nanoparticles coated with cationic compounds, especially PEI (Poly Ethylene Imine), chitosan, CTAB (Cetyl TrimethylAmmonium bromide), primary, secondary, tertiary or quaternary amine compounds, or from chitosan and its derivatives. Representative size and zeta potential of some cationic nanoparticles are given in FIG. 1.

In a preferred embodiment, the cationic nanoparticle used according to the invention is selected from cationic polysaccharide nanoparticles, especially from cationic maltodextrin nanoparticles such as porous maltodextrin with or without a lipid core nanoparticle (NP+ or DGNP, respectively), or from PLA or PGA or PLGA nanoparticles coated with cationic compounds, such as PEI, chitosan and its derivatives such as trimethyl-chitosan.

In a second aspect, the present invention relates to a combination product essentially consisting of cationic nanoparticles as defined above and live viruses, especially non-enveloped live viruses as defined above, its method of preparation and its uses.

1. The combination product is preferably obtained by incubating the viruses with cationic nanoparticles, especially with an excess of cationic nanoparticles. The quantity of cationic nanoparticles is at least 10 times, possibly 100 times or even 1000 times larger (in weight/weight) than the quantity of infectious virus particles. The apparent weight of the proteins can be determined by sensitive assay such as the microBCA method, or all other convenient methods. If the ratio corresponds to the relative number of cationic nanoparticles to the number of virus particles, the two components can be combined in a ratio 1:1, or 10:1 or even 100:1. Irrespective of the method of calculation of such ratio, the required quantity of cationic nanoparticles depends on the purity of the viruses; and the purity of the viruses is linked to the quantity of proteins which are naturally mixed with the viruses in viral preparation. It thus can be noted that the combination product may contains proteins, which are associated with viruses and is thus mentioned as “consisting essentially of cationic nanoparticles and live virus”. The more pure is the preparation, the lower is the need for a large amount of particle. The positive zeta potential of combination products obtained under these conditions suggests that cationic nanoparticles cover the viruses. Thus, in a particular embodiment, the viruses should be covered by cationic nanoparticles. However, the results do not allow to exclude that nanoparticles may interact with viruses in solution so that the nanoparticles facilitate the entry of the viruses, even if the viruses are not covered by the nanoparticles.
2.

Without wanting to be bound by any theory, the inventors believe that the excess of cationic nanoparticles leads nanoparticles to be adsorbed on the surface of viruses and thus seems to result in nanoparticle-coated viruses.

Accordingly, the present invention further relates to a method of preparing the combination product defined above, said method comprising a step of incubating live viruses with cationic nanoparticles, especially an excess of cationic nanoparticles according to the invention if needed.

In a particular embodiment, the incubation is advantageously carried out at 37° C. in a culture medium for at least 1 hour. The skilled person will chose the appropriate culture medium as a function of the virus to be incubated. Generally, appropriate culture medium include, without being limited thereto, Minimum essential medium and its modifications (Dulbecco modified, F12 based, ATCC modified . . . ), Eagle's medium and its modifications, RPMI 1640 medium and its modification, Iscove's Modified Dulbecco's Medium and its modification. The culture medium may contain additives like heat-inactivated serum supplementation (from fetal bovine, horse, calf . . . ), antibiotics, amino-acids, buffering reagents.

The combination product according to the invention is particularly interesting for several applications such as, without being limited thereto, vaccines, virus production, virus stabilization, gene therapy, oncotherapy, disease treatment or protein production. Compared to virus alone, the combination product according to the invention allows using smaller amounts of viruses for obtaining a given effect, the use of nanoparticles combined with live viruses in these viral applications is particularly advantageous.

Further, the combination provides protection of the virus against thermal denaturation (FIG. 12). It is proposed that the stability of the viral preparation is improved thanks to the presence of the nanoparticle coating. Thus, the invention also concerns the use of cationic nanoparticles to improve the stability of the viral preparation, in a range of temperature comprised between +1° C. and +45° C., preferably between +4° C. and +25° C., more preferably between +4° C. and +8° C. In a particular embodiment, the viruses combined with cationic nanoparticles are stable at room temperature, meaning temperatures generally between comprised between +15° C. and +27° C., more precisely around +20° C.

Thus, the present invention also relates to a combination product according to the invention as described above for use in a method of disease treatment. Particularly, the present invention relates to a method for treating a disease in a patient, said method comprising a step of administering a pharmaceutically effective amount of the combination product according to the invention to a patient in need thereof.

In a particular embodiment, the combination product according to the invention may be used in a method for treating cancer based on the use of an oncolytic virus. Oncolytic viruses are able of selectively inducing the lysis of cancerous cells. In other terms, the invention also relates to a method of treating cancer comprising administering the combination product according to the invention to a patient in need thereof. Virus-mediated oncotherapy is a widespread method wherein viruses are used for treating cancer by specifically targeting and destroying cancerous cells. This method relies on the ability (either naturally or because genetically modified to do so) of some viruses to only replicate in cancerous cells. The skilled person in the art knows several oncolytic non-enveloped viruses and knows how to select them (see for instance Meerani et al., European Journal of Scientific Research, 2010, 40(1), 156-171; or Bartlett et al. Molecular Cancer 2013, 12:103). Thus, in this aspect of the invention, the cationic nanoparticles may be combined with an oncolytic non-enveloped virus. Typically, oncolytic non-enveloped viruses are for example: parvoviruses or adenoviruses, such as hTERT-Ad and Ad5/3-D24-GMCSF, as disclosed in the review Bartlett et al. Molecular Cancer 2013, 12:103.

The combination product according to the invention may also be used as a vaccine or in a vaccine composition in a so-called “viral immunotherapy”. A vaccine composition, once it has been administered to a subject, elicits a protective immune response against the one or more antigen(s) which is (are) comprised herein. It induces a protective immune response against, for example, a microorganism, to efficaciously protect the subject against infection. Thus, by using a cationic nanoparticle combined with non-enveloped virus as antigen, lower quantities of viruses are needed for obtaining a desired immune-response. Typically, for the vaccination, the viruses used are live viruses attenuated or not, defective or not, recombinant or not.

Further, the combination product according to the invention may be used as a gene therapy composition. Gene therapy relies on the replacement of viral genes by therapeutic genes in order to deliver such genes to target cells. Combining the viral vector with nanoparticles will allow the virus to efficiently enter the target cells. Thus such combination will improve the efficacy of the treatment. Typically, for the gene therapy, the viruses used are recombinant.

The present invention also relates to a method for producing viruses by using cationic nanoparticles combined with live viruses, especially non-enveloped viruses according to the invention. Viruses cannot support their replication by their own and necessitate living hosts to do so. Viral production necessitates incubating the virus with living cells to allow the virus to replicate by using the cell machinery, and collect the viruses produced thereof. The viruses are produced either by escaping from the cell by viral shedding or released by the lysis of the host cell. The use of cationic nanoparticles combined with live viruses, especially non-enveloped viruses improves entry of the viral particles into the host cells, which potentiates the production of the virus and thus increases viral production yield.

According to the invention, the method for producing viruses comprises the steps of:

a) Incubating a host cell culture with a combination product according to the invention; and
b) Harvesting the viruses produced by the host cell.

In one embodiment, incubation step a) is carried out at 37° C. in a culture medium during at least one hour. Suitable culture media include, without being limited thereto, Minimum essential medium and its modifications (Dulbecco modified, F12 based, ATCC modified . . . ), Eagle's medium and its modifications, RPMI 1640 medium and its modification, Iscove's Modified Dulbecco's Medium and its modification. The culture medium may contain additives like heat-inactivated serum supplementation (from fetal bovine, horse, calf . . . ), antibiotics, amino-acids, and/or buffering agents.

The present invention also relates to any recombinant virus that can be used for improving the production of recombinant proteins. For example, the use of combination product wherein the virus is, but not limited to, a baculovirus for the production of recombinant protein. Baculovirus, from Baculoviridae family, are enveloped virus infecting insect cells, widely used for production of foreign protein. In the scope of the present invention, the combination of recombinant baculoviruses with cationic nanoparticles should allow to improve or optimize the infection of insects cells and thus to improve the protein production capacity. In addition to Baculoviridae, other viruses can be used for protein production such as Caulimoviridae viruses for protein production in plants.

The invention further relates to a pharmaceutical composition comprising the combination product containing cationic nanoparticles and live viruses, especially non-enveloped virus according to the invention and at least one pharmaceutically acceptable excipient. Said excipients are chosen according to the pharmaceutical form and administration mode required, among the normal excipients that are known to persons skilled in the art.

### FIGURES

FIG. 1: Example of size (Z-Average), poly-dispersity index (PDI) and Zeta potential of cationic nanoparticles.

PLGA PEI: PLGA nanoparticles coated with PEI. PLGA Chitosan: PLGA nanoparticles coated with Chitosan. NP+: cationic maltodextrin nanoparticle. DG70: NP+ with lipid core. Liposome +: Cationic liposome. PLGA: Poly(Lactic-co-Glycolic Acid). PEI: Poly(Ethylenelmine).

FIG. 2: Example of size and Zeta potential of DG70 cationic nanoparticle, viruses and combination products at the mass ratio 1/3.

DG70: cationic maltodextrin nanoparticle with lipid core. GMB: Gumboro virus, NDV: Newcastle Disease Virus, Polio: Poliovirus-1, Reo: Reovirus, Rota: Rotavirus SA-11, BVDV: Bovine viral diarrhea virus, RSV: Respiratory syncytial virus, HSV: Herpes Simplex Virus 1.

FIG. 3: Example of size and Zeta potential of DG70 cationic nanoparticle, killed viruses and combination products at the ratio 1/10 (w/w). DG70: cationic maltodextrin nanoparticle with lipid core. GMB: Gumboro virus, Polio: Poliovirus-1, Reo: Reovirus, Rota: Rotavirus SA-11.

FIG. 4: Fold induction of UV-inactivated virus transfection alone or in combination product. PLGA PEI: PLGA nanoparticles coated with PEI. PLGA Chitosan: PLGA nanoparticles coated with Chitosan. NP+: cationic maltodextrin nanoparticle. DG70: NP+ with lipid core. Liposome +: Cationic liposome. PLGA: Poly(Lactic-co-Glycolic Acid). PEI: Poly(EthyleneImine). Polio: Poliovirus-1, HSV: Herpes Simplex Virus, BVDV: Bovine Viral Diarrhea Virus, RSV: Respiratory Syncitial Virus, Rota: Rotavirus, Reo: Reovirus, NDV: Newcastle Disease Virus, GMB: Gumboro virus.

FIG. 5: Study of chlorpromazine (CPZ) on gumboro associated or not with NP on killed virus endocytosis.

FIG. 6: CPE of DG70 cationic nanoparticles, poliovirus-1 and the related combination products. Hep-2 cells were infected with various dilutions of poliovirus-1, alone or in combination with nanoparticles, ranging from 10⁵ to 10⁻⁴ TCID50/mL. The CPE was evaluated after 6 days. Data are from 2 independent experiments and are expressed as mean+SD. cells: untreated cells, nano: cationic maltodextrin nanoparticle with lipid core (DG70).

FIG. 7: Kinetics of the viral titer in cells infected with poliovirus-1 alone or in combination with DG70-nanoparticles at various virus TCID50/ml. Hep-2 cells were infected with various dilutions of poliovirus-1, alone or in combination product, ranging from 10¹ to 10⁻⁴ TCID50/mL. Supernatants were collected at different times post-inoculation. Poliovirus titer was determined by limiting dilution assay for 50% tissue culture infection doses in Hep2 cell cultures by the method of Reed-Muench.

FIG. 8: Fold increase of the infectious capacity of virus alone versus the combination products. Supernatant of infected cells with virus alone or combination products were used to re-infect cells. The CPE were calculated and the fold increase between virus and combination products were expressed as log 10. PLGA(−): uncoated anionic PLGA nanoparticles. PLGA Chitosan: PLGA nanoparticles coated with Chitosan. PLGA PEI: PLGA nanoparticles coated with PEI. NP+: cationic maltodextrin nanoparticle. DG70: NP+ with lipid core. Liposome +: Cationic liposome. PLGA: Poly(Lactic-co-Glycolic Acid). PEI: Poly(Ethylenelmine). Polio: Poliovirus-1, CPV: Canine ParvoVirus. Rota: Rotavirus SA-11, HSV: Herpes Simplex Virus 1, RSV: Respiratory Syncitial Virus, BVDV: Bovine viral diarrhea Virus. Tox: Cell toxicity. Inhib: Inhibitory effect of the combination product compared to the virus alone.

FIG. 9: Percentage of VP-1 positive cells after infection for 6 or 18 hours with poliovirus-1 or DG70-poliovirus-1 combination products. Numbers express the total number of cells/the number of VP1+ cells (% of VP1+ cells). DG70: cationic maltodextrin nanoparticle with lipid core. Polio: Poliovirus-1. MOI: Multiplicity of Infection. Percentages refer to infected cells. Grey blocks refer to the presence of apparent lysis plaques.

FIG. 10: Representative microphotography of the detection of virus by VP1 immunofluorescence. Cells were infected at a multiplicity of infection of 0.6 for 18 h and anti-VP1 immunostaining (lower circles) was performed. Nucleus are stained by Hoescht (upper circles).

FIG. 11: Detection of viral RNA by Q-PCR. Cells were infected at a multiplicity of infection of 7.10⁻² and RNA were collected after 48 h. Numbers express the absolute value of Ct relative to the common non-coding region of enteroviruses. DG70: cationic maltodextrin nanoparticle with lipid core. Polio: Poliovirus-1.

FIGS. 12A, 12B: Study of the stabilization of virus against thermal denaturation. A: 2 h30 at 55° C.; B: 24 h at 45° C.

### EXAMPLES

### Material and Methods

1/ Synthesis of Nanoparticles:
Cationic maltodextrin nanoparticles: Maltodextrins are dissolved in 2 N sodium hydroxide with magnetic stirring at room temperature. Addition of epichlorhydrin and GTMA yields a cationic polysaccharide gel that is then neutralized with acetic acid and crushed using a high pressure homogenizer (Emulsiflex C3, France). The nanoparticles thus obtained are purified by tangential flow ultra-filtration (Centramate Minim II, PALL, France) using a 300 kDa membrane (PALL, France).
DG70 nanoparticles: Porous maltodextrin-based with lipid core nanoparticles (DGNP) were prepared as described previously (Patent WO2014041427). The cationic maltodextrin nanoparticles obtained as described above are mixed with dipalmitoyl phosphatidyl glycerol (DPPG) above the gel-to-liquid phase transition temperature to produce DG70.
Anionic PLGA nanoparticles: Negative PLGA nanoparticles (PLGA(−)) are produced by nanoprecipitation (Le Broc-Ryckewaert D et al., Int J Pharm., 2013). The PLGA copolymer is dissolved in acetone/ethanol (85:15) mixture composing the organic phase then injected in aqueous phase under stirring. Organic solvents are eliminated by vacuum evaporation.
Cationic PLGA coated with PEI: PLGA nanoparticles are produced by nanoprecipitation. The PLGA copolymer is dissolved in acetone/ethanol (85:15) mixture composing the organic phase. These nanoparticles are cationised by injecting the dissolved PLGA copolymer in aqueous phase supplemented with 10% (w/w) Polyethylenimine (PEI) under stirring. Organic solvents are eliminated by vacuum evaporation.
Cationic PLGA coated with Chitosan: PLGA nanoparticles are produced by nanoprecipitation. The PLGA copolymer is dissolved in acetone/ethanol (85:15) mixture composing the organic phase. These nanoparticles are cationised by injecting the dissolved PLGA copolymer in aqueous phase supplemented with 10% (w/w) Chitosan solution under stirring. Organic solvents are eliminated by vacuum evaporation.
Cationic liposomes: DPPC/DPPE (1,2-dipalmitoyl-sn-glycero-3-phosphocholine/1,2-dipalmitoyl-sn-glycero-3-phosphocholine) liposomes are prepared by solubilizing DPPC and DPPE in ethanol, the solution is then injected with a syringe in water under stirring at 80° C. Liposomes are then purified by filtration, residual ethanol is eliminated under vacuum.

2/ Cell Lines

Hep-2 cell line: Hep-2 cells were provided by BioWhittaker (Vervier, Belgium). The cell line, well adapted for enteroviruses culture, was grown in Eagle's minimum essential medium (MEM) supplemented with 10% inactivated fetal bovine serum (FBS), 1% L-glutamin and penicillin (100 U/ml)-streptomycin (100 mg/ml) and fungizone (0.25 mg/ml; Invitrogen, Saint Aubin, France) in an atmosphere of 5% CO2 and a humidified air at 37° C.

CMT-U27 cell line: Canine mammary tumor (CMT-U27) cell line was derived from a primary tumor (infiltrating ductal carcinoma). CMT-U27 cell line (a gift from Associated Professor Eva Hellmen) was obtained from the Uppsala University, Sweden, cultured in RPMI 1640 medium supplemented with 10% heat-inactivated fetal bovine serum, 1% L-glutamine, penicillin-streptomycin (50 IU/mL) in an atmosphere of 5% CO2 and a humidified air at 37° C. These cells are used to produce the Canine Parvovirus.

CRFK cell line: Monolayers of Crandell Rees Feline Kidney (CRFK) cells (ATCC® no. CCL-94™) were grown in Dulbeco™ Minimum Essential Medium (MEM) supplemented with 10% Fetal Bovine Serum (FBS), 1% penicillin and streptomycin and non-essential amino acids at 37° C. and 5% CO₂. This cells are used to product the Canine Parvovirus.

Vero cell line: These cells were provided by ATCC (ATCC® CCL-81™). The cell line, was grown in Dulbecco's Modified Eagle Medium (DMEM) supplemented with 10% inactivated fetal bovine serum (FBS), 1% L-glutamine and 1% penicillin Streptomycin in an atmosphere of 5% CO2 and a humidified air at 37° C.

MA 104 cell line: cells were provided by ATCC (ATCC® CRL-2378.1™). The cell line, was grown in Eagle Minimum Essential Medium (MEM) supplemented with 10% inactivated fetal bovine serum (FBS), 1% L-glutamine and 1% Penicillin-Streptomycin in an atmosphere of 5% CO2 and a humidified air at 37° C.

MDBK cell line: cells were provided by ATCC (ATCC® CCL-22™). The cell line, was grown in Eagle Minimum Essential Medium (MEM) supplemented with 10% horse serum (HS), 1% L-glutamine, 1% non-essential amino acids and 1% Penicillin-Streptomycin in an atmosphere of 5% CO2 and a humidified air at 37° C.

Raw 264.7 cell line: The Raw cells (ATCC® TIB-71™) are macrophage cells of Mus musculus. The cell line was grown in Dulbecco's Modified Eagle Medium (DMEM) supplemented with 10% inactivated fetal bovine serum (FBS), 1% L-glutamine, 1% non-essential amino acids and 1% penicillin Streptomycin in an atmosphere of 5% CO2 and a humidified air at 37° C.

3/ Live Viruses

Poliovirus-1: The monovalent oral poliovirus (Poliovirus 1) used in this example was provided by Eurovir Hygiene-Institut (Luckenwalde, Germany) with a virus titer at 10⁶ TCID50/mL and stored at −20° C.

Canine Parvovirus: Canine parvovirus (CPV), a single strand DNA virus and a significant worldwide canine pathogen belonging to the family Parvoviridae, is a highly contagious and a principal etiological agent of hemorrhagic enteritis in dogs. The strain used in this study was from ATCC (ATCC® VR-2017™) and was grown in CRFK cells and CMT-U27 cells.

Rotavirus: Simian rotavirus (ATCC® VR-1565™) strain SA-11, is a double stranded RNA virus of the Reoviridae family. Rotavirus causes diarrheal disease in children. The recommended hosts are MA-104 (ATCC® CRL-2378-1).

Human herpesvirus type 1: Herpes simplex virus type 1 (HSV-1) is a member of the family Herpesviridae, that infects humans. This is an enveloped DNA virus. The strain used in this study was from ATCC (ATCC® VR-733™). The host cells are Vero cells (ATCC® CCL-81™)

Bovine viral diarrhea virus: BVDV (NBL2) causes one of the most significant infectious diseases in the livestock industry worldwide due to its high prevalence, persistence and clinical consequences. BVDV is single-stranded RNA enveloped viruses. The strain used in this study was from ATCC (ATCC® VR-534™). The host cells are MDBK cells (ATCC® CCL-22™)

Murine norovirus: Murine norovirus S99 Berlin (MNV) is a species of norovirus affecting mice. It is a non-enveloped virus with a linear positive-sense RNA genome. The host cells are Raw 264.7 cells (ATCC® TIB-71™).

Human respiratory syncytial virus: Respiratory syncytial virus (RSV) of the family Pneumoviridae causes respiratory tract infections during infancy and childhood. It is an enveloped virus, single-stranded RNA. The host cells are Hep-2 cells (BioWhittaker, Vervier, Belgium).

4/ Killed Virus

Killed virus are virus that has been inactivated and did no longer show infectious capacity.

Viruses were produced in cell lines as described in section “3/ Live Virus” and were UV-inactivated for 30 min under the Microbiological safety workbench (UV lamp). The size and the purity of the virus is determined with the Zetasizer Nano ZS. Gumboro, Newcastle Disease Virus (NDV) and Reovirus purified killed viruses were kindly provided by Intervet (MSD Sante Animale, France).

5/ Labeling of Killed Virus

Killed virus are covalently labeled with fluorescein. Viral protein concentration is determined by the microBCA method. Briefly, 1 mg of FITC (Fluorescein IsoThioCyanate, dissolved in anhydrous DMSO) was added to 10 mg of viral proteins solubilized in 0.1M bicarbonate buffer (pH 9.5), and the solution was mixed for 6 h in the dark at room temperature. The preparation was purified by gel filtration on a PD-10 Sephadex desalting column (Sigma-Aldrich) and exclusion fractions were collected.

6/ Combination Product

The combination of cationic nanoparticles and viruses is carried out by mixing both components in a relevant culture medium for the test on cell lines.

7/ Size and Zeta Potential Analysis

The hydrodynamic diameter of cationic nanoparticles, viruses or the combination products was measured in 15 mM NaCl by dynamic light scattering using a Zetasizer Nano-ZS instrument (Malvern Instruments, Orsay, France). The zeta potentials of nanoparticle preparations were determined in water (ZetaSizer NanoZS analyzer, Malvern Instrument).

8/ Cytometry Analysis on Killed Viruses

Cells were treated with the combination products (section 6 of the Material and Methods) comprising fluorescently labelled killed viruses. After 3 hours, cells were collected and cell fluorescence was analysed on an Accuri c6 flow cytometer (BD Biosciences, Erembodegem, Belgium).

9/ CPE

Cytopathogenic effect (CPE) is a structural change in host cells that are caused by viral infection. The infecting virus causes lysis of the host cell through changes in cell morphology. Common examples of CPE include rounding of the infected cell, fusion with adjacent cells to form syncytia, and the appearance of nuclear or cytoplasmic inclusion bodies. CPE were determined using an inverted microscope.

10/ Progeny and Virus Titration

Viruses were serially diluted in presence or not of nanoparticles in the relevant medium from 10⁻¹ to 10⁻¹² in eight replicates in 96-well plates. Then cells were incubated for 5 days in a 5% CO2 atmosphere at 37° C. Afterwards the plates were examined using an inverted microscope to evaluate the extent of the virus-induced cytopathic effect in the cell culture (CPE). Calculation of estimated virus concentration was carried out by the Spearman-Kärber method and expressed as log₁₀ TCID₅₀. Supernatants from each well were collected and used to infect naïve cells. The progeny test is performed to determine the MOI (Multiple of infection=number of infecting virus per one cell). The progeny is a virus titration. After several days of incubation, the virus have infected the cells and have produced a virus titer (TCID50/mL).

11/ Determination of Viral RNA Content by Q-RT PCR

Poliovirus positive strand RNA was quantitated by QRT-PCR. Total RNA was extracted with Tri-Reagent® (Sigma-Aldrich) following manufacturer's instructions. Total RNA was measured by a quantitative RT-QPCR for RNA with the Affinity script QPCR cDNA synthesis kit and the brilliant II QPCR kit (Agilent technology, France). Positive strand specific RT was carried out on extracted RNA by using the reverse primer at 42° C. for 15 min. PCR was performed with universal cycle conditions (10 min at 95° C., 40 cycles of 30 s at 60° C.) on a Mx3000p (Agilent technology, France). The following primers, used to detect Poliovirus RNA, were located within the enterovirus 5′-nontranslated region, which is highly conserved among enterovirus serotypes: forward (5′-CCC TGA ATG GGG CTA ATC), reverse (5′-ATT GTC ACC ATA AGC AGC CA) and probe (5′-VIC-AAC CGA CTA CTT TGG GTG TCC GTG TTT-TAMRA) (Applied Biosystems, ThermoFisher Scientific, France). Results were expressed as cycle threshold (Ct) which is inversely proportional to RNA level.

12/ Determination of Viral Protein Content by Immunofluorescence

After washing with PBS, Hep-2 cells infected by Poliovirus were fixed with fresh 4% paraformaldehyde and permeabilized with chilled methanol/acetone. Nonspecific sites were blocked with rabbit serum/anti-Fc receptor solution (Miltenyibiotec®). Cells were first labelled with primary antibodies, mouse anti-enterovirus VP1 anti-body (clone 5D8/1 Dako®), then with rabbit anti-mouse alexa Fluor 488 (Molecular Probes®). Nuclei were stained by Hoescht dye solution (Sigma, France). Slides were mounted and visualized by using a Zeiss LSM 710 confocal laser-scanning microscope equipped with argon and helium-neon lasers.

13/ Evaluation of Clathrin Endocytosis of Gumboro Virus Associated or not with DG70

Vero cells were treated with the combination products (section 6 of the Material and Methods) comprising fluorescently labelled killed viruses. Cells were treated with or without 15 μg/ml of chlorpromazine for 3 hrs. Cells were then collected and cell fluorescence was analysed on an Accuri c6 flow cytometer (BD Biosciences, Erembodegem, Belgium).

### Results

1/ Cationic Nanoparticles Synthesis and Characterisation

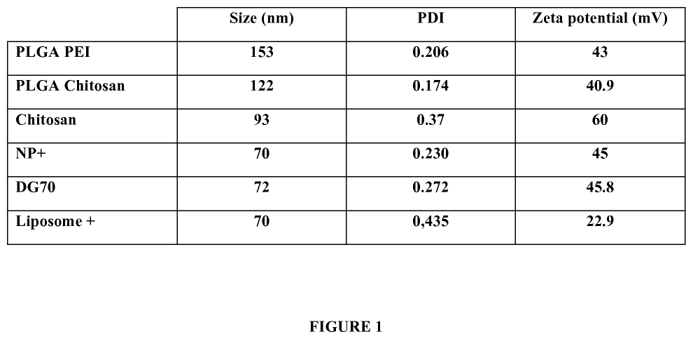

Different cationic nanoparticles were synthetized and analysed by dynamic light scattering. Among produced cationic nanoparticles: PLGA coated with PEI or Chitosan, cationic maltodextrin (NP+) and with lipid core (DG70), cationic liposome or chitosan. Results are depicted in FIG. 1.

2/ Study on Killed Virus

a/ Combination product: Formulation of cationic nanoparticle and virus UV-inactivated virus (killed virus) were associated to cationic nanoparticles and the size and the zeta potential of the resulting combination products, so called formulations, were determined by dynamic light scattering.

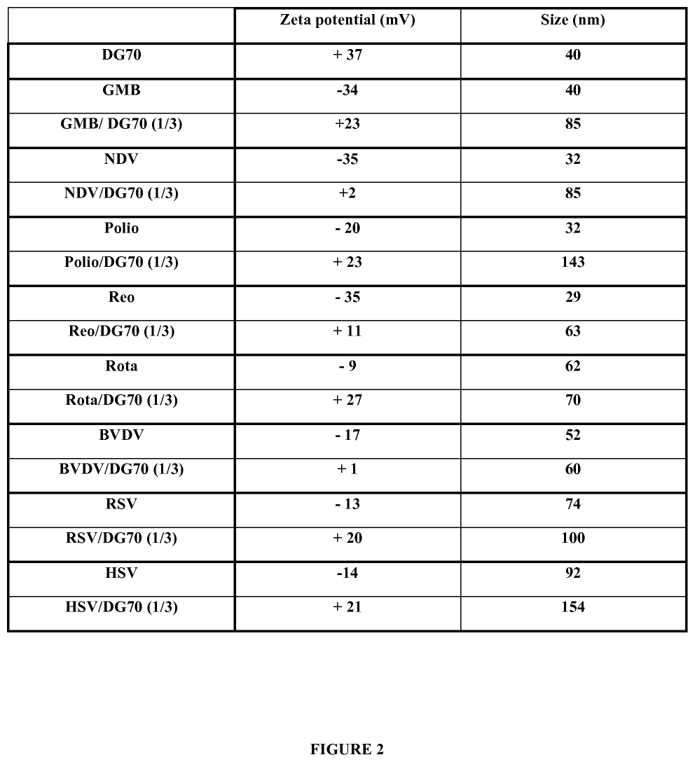

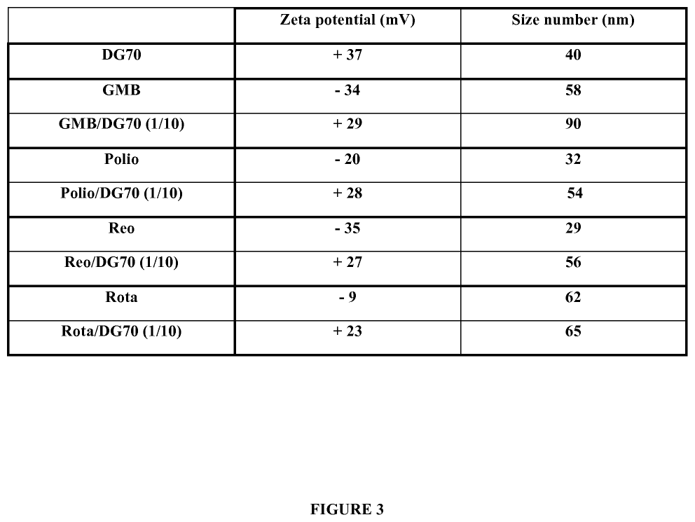

Formulations with 3 times more nanoparticles (1/3 mass ratio, see FIG. 2) or with 10 times more nanoparticles (1/10 mass ratio, see FIG. 3) than viruses were analysed. The combinations have a greater size compared to cationic nanoparticles alone or virus alone which confirms the association. In addition, the zeta potentials of the viruses are negative while the combination products are positive, suggesting that cationic nanoparticles cover the virus.

In live virus experiments (section 3), the ratio virus/cationic nanoparticles is at least 1/1000 reinforcing the coating of viruses by cationic nanoparticles.

b/ Transfection of Killed Viruses

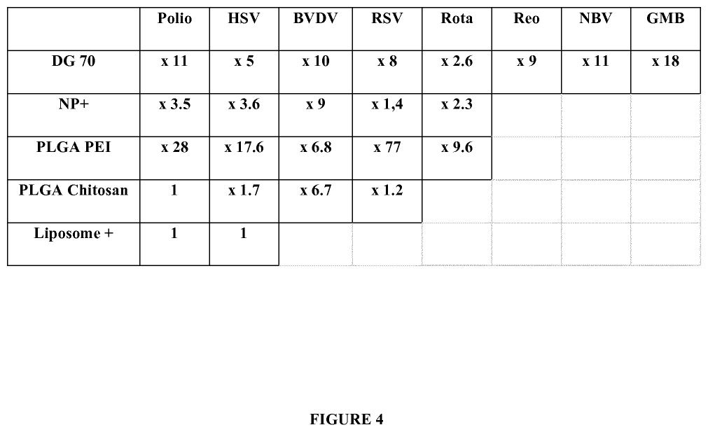

Combination products of cationic nanoparticle with UV-inactivated and fluorescently-labeled viruses were produced. The cationic nanoparticles used in this study were mainly DG70 nanoparticles. Killed virus (5 μg) and cationic nanoparticles (15 μg) were added to a medium with 10% serum before incubation with cells. The amount of viruses in the cells was analyzed by flow cytometry and representative data are summarized in FIG. 4.

Compared to UV-inactivated virus alone, the combination products, according to the invention, highly increase the virus entry into the cells. This could increase the infectious capacity of a live virus.

c/ Mechanisms of Endocytosis:

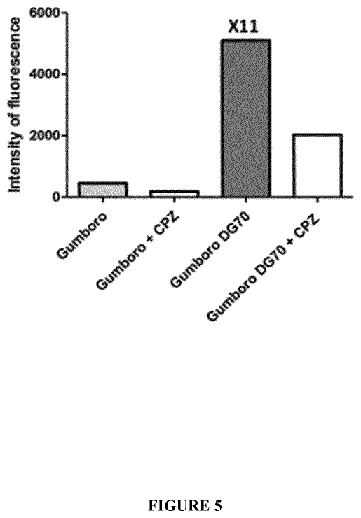

The endocytosis of gumboro virus was evaluated by FACS in presence of a clathrin inhibitor (chlorpromazine) after 3 h of incubation in Vero cells. The FIG. 5 is a representative study of virus endocytosis where we found that cationic nanoparticle mainly increases the virus endocytosis via the clathrin pathway, same results were obtained with all the virus tested.

3/ Study on Live Viruses

a/ Effect on CytoPathogenic Effect

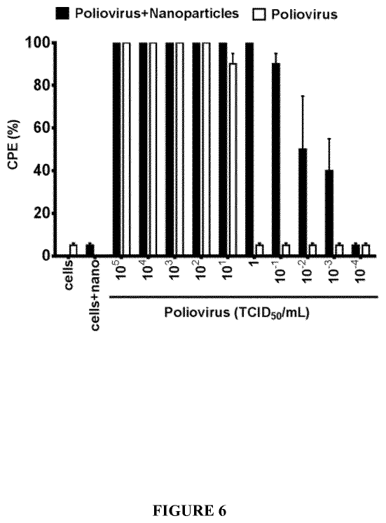

The cytopathogenic effect (CPE) is defined as the change in cell structure and viability due to a viral infection, typically a lysis plaque. The CPE of cationic nanoparticles, viruses or combination products are analysed with an inverted microscope. As described in FIG. 6, cationic nanoparticles increase the CPE of the virus by 4 log 10 TCID50/ml meaning a higher efficacy of at least 10000.

b/ Effect on Virus Production

i/ Effect on Progeny of Poliovirus

The viral shedding refers to the expulsion and release of virus progeny following successful reproduction during a host-cell infection. This allows to determine the amount of infectious viruses and their capacity of using the cellular machinery to reproduce themselves. A critical step is the entry into the cells.

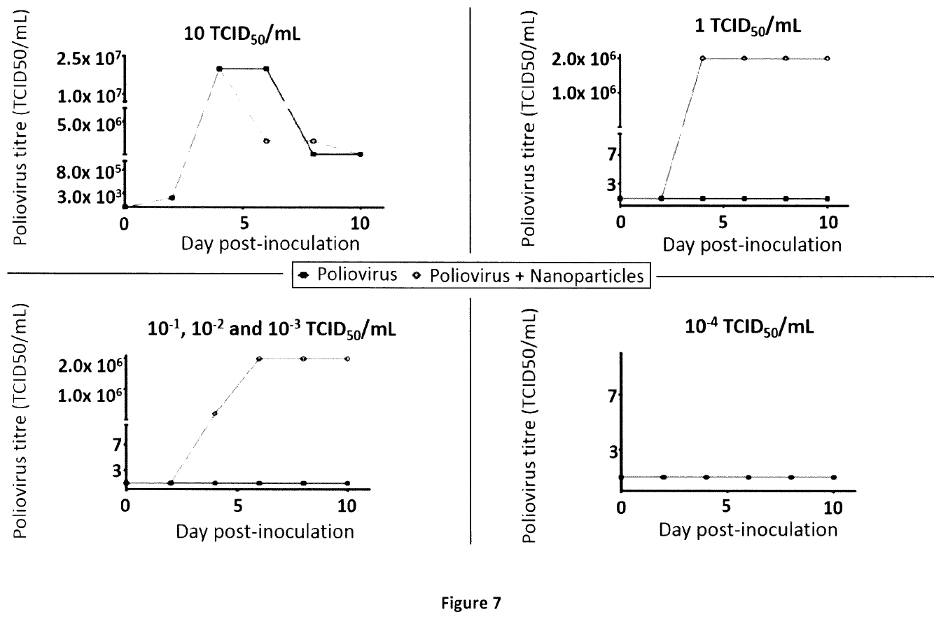

To compare the infectious capacity of viruses versus the related combination products (nanoparticles combined with virus), the cells were infected with viruses alone or viruses combined with cationic nanoparticles (first round). Then the supernatants of the cells were collected and reused to infect non-infected cells (second round). The lysis of the later cells reveals the infectious capacity of the viruses or the combination products. As described in FIG. 7, the capacity of infectious of poliovirus is increased by several log 10 compared to the viruses alone. For example, the poliovirus-1 combined with DG70 cationic nanoparticles is 3 to 4 log 10 (=1000 to 10000) more infectious than the poliovirus-1 alone.

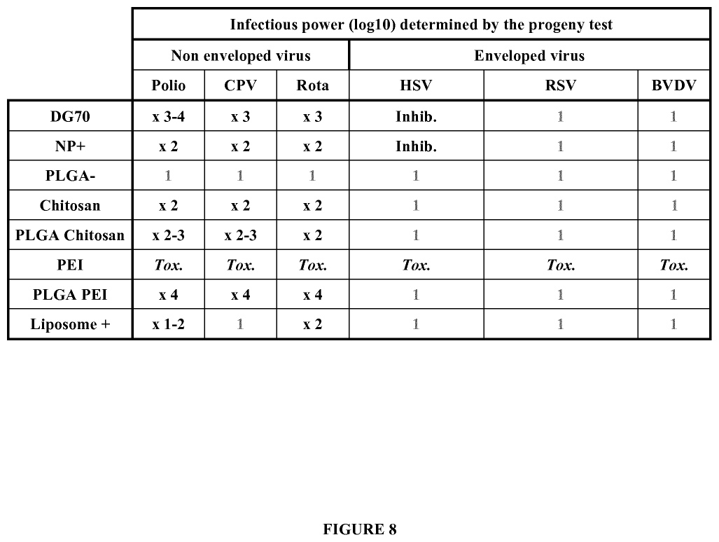

In FIG. 8 we summarize the increase of the infectious capacity observed by different combinations of cationic nanoparticles and non-enveloped virus. Poliovirus-1, Canine ParvoVirus and Rotavirus SA-11 combined with PLGA PEI cationic nanoparticles are 4 log 10 more infectious than viruses alone while the PLGA PEI cationic nanoparticles does not increase the infection capacity of enveloped virus. Then, an uncoated anionic PLGA nanoparticle was tested and no increase of the virus infectious capacity was observed.

The results confirm that the use of cationic nanoparticles increase the infectious capacity of non-enveloped live viruses.

iii/ Effect on Viral RNA and Protein Productions

In order to confirm the increase in the production of virus, measures of viral RNA and protein was performed by Q-PCR and immunofluorescence against VP1, one of the main protein of poliovirus-1.

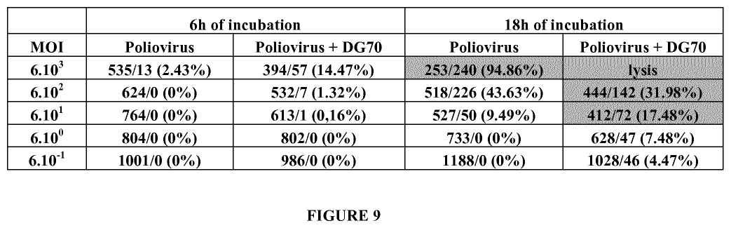

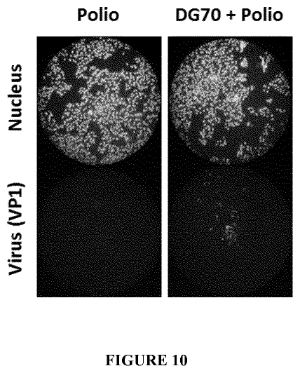

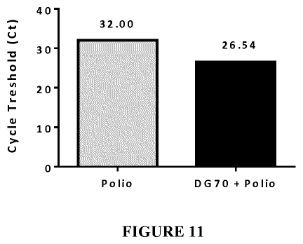

Detection of VP1 protein compared to virus alone showed that, the DG70 combination product induced a higher and earlier viral expression (FIG. 9 and FIG. 10). At the nucleic acid level, the detection of the common non-coding region of enteroviruses RNA revealed a decrease of 5.46 cycle threshold by virus alone and the related DG70 cationic nanoparticle combination product. This corresponds to a 44-fold increase in the viral RNA production (FIG. 11) and means that cationic nanoparticles enhance the production of viruses at the protein and nucleic acid levels.

iv/ Study of Virus Protection Against Thermal Denaturation

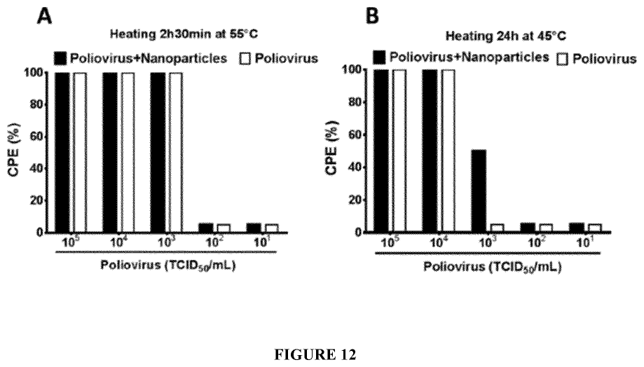

The ability of cationic nanoparticles to protect viruses from thermal denaturation has been tested. Polioviruses were incubated either at 45° C. for 24 h or at 55° C. for 2 h30 in presence or not of DGNP. At 55° C. after 2 h30 incubation no protection was observed even in presence of DGNP (FIG. 12,A), while we observed a partial protection with NP at doses of virus corresponding at 1000 TCID50/ml when viruses were combined with nanoparticles at 45° C. for 24 h (FIG. 12,B). This result suggests that the combination provides protection of the virus against thermal denaturation.

1. Use of cationic nanoparticles for enhancing the infectious capacity of live virus.

2. The use according to claim 1, wherein said virus is selected from the group consisting of Adenoviridae, Caulimoviridae, Rudiviridae, Papillomarividae, Phycodnaviridae, Tectiviridae, Papovaviridae, Circoviridae, Parvoviridae, Birnaviridae, Reoviridae, Astroviridae, Caliciviridae, Picornaviridae, Potyviridae, Poliomarividae, Hepeviridae, Arteriviridae, Anelloviridia, Papillomarividae, Paramyxoviridae, Togaviridiae, Herpesviridae, Orthomyxoviridae, Flaviviridae, Hepadnaviridae, Rhabdoviridae, Poxviridae, Filoviridae, Retroviridae, Coronaviridae, Baculoviridae, Reoviridae and phages and bacteriophages and their combinations.

3. The use according to claim 1, wherein said virus is a recombinant or a defective or an attenuated virus.

4. The use according to claim 1, wherein said virus is a non-enveloped virus.

5. The use according to claim 4, wherein said non-enveloped virus is selected from the group consisting of Adenoviridae, Caulimoviridae, Myoviridae, Siphoviridae, Podoviridae, Rudiviridae, Papillomarividae, Phycodnaviridae, Tectiviridae, Papovaviridae, Circoviridae, Parvoviridae, Birnaviridae, Reoviridae, Astroviridae, Caliciviridae, Picornaviridae, Potyviridae, Poliomarividae, Hepeviridae, Arteriviridae, Anelloviridiae and their combinations.

6. The use according to claim 1, wherein the cationic nanoparticles cover the virus.

7. The use of cationic nanoparticles to improve the stability of the viral preparation, in a range of temperature from +1° C. to 45° C.

8. The use according to claim 1, wherein said nanoparticles are selected from cationic polysaccharide nanoparticles, from PLA or PGA or PLGA nanoparticles coated with cationic compounds, from chitosan and its derivatives or from cationic micelles or cationic liposomes.

9. The use according to claim 8, wherein said nanoparticles are selected from cationic polysaccharide nanoparticles, especially from cationic maltodextrin nanoparticles such as porous maltodextrin with or without a lipid core nanoparticle such as NP+ or DGNP, or from PLA or PGA or PLGA nanoparticles coated with cationic compounds, such as PEI, chitosan and its derivatives, such as trimethylamoniumchitosan.

10. A combination product consisting essentially of cationic nanoparticles and live virus.

11. The combination product according to claim 10, wherein said virus is selected from the group consisting of Adenoviridae, Caulimoviridae, Rudiviridae, Papillomarividae, Phycodnaviridae, Tectiviridae, Papovaviridae, Circoviridae, Parvoviridae, Birnaviridae, Reoviridae, Astroviridae, Caliciviridae, Picornaviridae, Potyviridae, Poliomarividae, Hepeviridae, Arteriviridae, Anelloviridia, Papillomarividae, Paramyxoviridae, Togaviridiae, Herpesviridae, Orthomyxoviridae, Flaviviridae, Hepadnaviridae, Rhabdoviridae, Poxviridae, Filoviridae, Retroviridae, Coronaviridae, Baculoviridae, Reoviridae and phages and bacteriophages and their combinations.

12. The combination product according to claim 11, wherein said nanoparticles are selected from cationic polysaccharide nanoparticles, from PLA or PGA or PLGA nanoparticles coated with cationic compounds, from chitosan and its derivatives or from cationic micelles or cationic liposomes.

13. The combination product according to claim 12, wherein said nanoparticles are selected from cationic polysaccharide nanoparticles, especially from cationic maltodextrin nanoparticles such as porous maltodextrin with or without a lipid core nanoparticle such as NP+ or DGNP, or from PLA or PGA or PLGA nanoparticles coated with cationic compounds, such as PEI, chitosan and its derivatives such as trimethyl-chitosan.

14. The use of a combination product as defined in claim 10 in a method of virus production.

15. A method for producing viruses comprising the steps of:
a) incubating a host cell culture with a combination product as defined in claim 10;
b) harvesting the viruses produced by the host cell.

16. The method of preparing the combination product as defined in claim 13, said method comprising a step of incubating live viruses with an excess of cationic nanoparticles, wherein said excess means that the quantity of cationic nanoparticles is preferably at least 10 times larger than the quantity of infectious virus particles (ratio weight/weight).

17. The combination product according to claim 10, for use in a method for treating cancer, cardiovascular diseases, neurodegenerative disorders and infectious diseases.

18. The combination product according to claim 10, for use as a vaccine.

19. The combination product according to claim 10, for use in gene therapy.

20. The use of the combination product according to claim 10, in a method of protein production.

21. A pharmaceutical composition comprising the combination product as defined in claim 10 and at least one pharmaceutically acceptable excipient.
